(19) United States
(12) Patent Application Publication
(10) Pub. No.: US 2012/0034296 A1
(43) Pub. Date: Feb. 9, 2012

(54) PROLONGED DURATION LOCAL ANESTHESIA WITH MINIMAL TOXICITY

(75) Inventors: Hila Epstein-Barash, Brookline, MA (US); Daniel S. Kohane, Newton, MA (US)

(73) Assignees: Massachusetts Institute of Technology; The Children's Medical Center Corporation

(21) Appl. No.: 13/263,804

(22) PCT Filed: Apr. 6, 2010
(86) PCT No.: PCT/US10/30061

§ 371 Date: Oct. 10, 2011

Related U.S. Application Data
(60) Provisional application No. 61/167,800, filed on Apr. 8, 2009.

(51) Int. Cl. A61K 31/529; A61P 23/02; A61K 31/573; A61K 31/58; A61K 9/127; A61K 31/519

(52) U.S. Cl. 424/450; 514/257; 514/267; 514/171

(57) ABSTRACT

Compositions containing site 1 sodium channel blockers for use as local anesthetics with rapid nerve block, improved potency and efficacy, and no local toxicity have been developed. Liposomes were employed for increased loading of the site 1 sodium channel blocker, producing prolonged duration of block without systemic toxicity. In one embodiment, the compositions contain a site 1 sodium channel blocker alone. In another embodiment, the compositions contain a site 1 sodium channel blocker in combination with a corticosteroid. As demonstrated by the examples, encapsulating site 1 sodium channel blockers in liposomes results in rapid and prolonged nerve block without systemic toxicity, which is enhanced by the addition of a corticosteroid. Fluid liposomes showed more rapid release of STX than did solid ones, and dexamethasone accelerated STX release.

### CROSS-REFERENCE TO RELATED APPLICATIONS

This application claims priority under 35 U.S.C. 119 to U.S. Ser. No. 61/167,800 filed Apr. 8, 2009.

### GOVERNMENT SUPPORT

This invention was made with government support under Grant No. GM073626 awarded by National Institute of General Medical Sciences. The government has certain rights in the invention.

### FIELD OF THE INVENTION

This relates generally to methods and compositions enhancing nerve blockade with local anesthetics.

### BACKGROUND OF THE INVENTION

The development of local anesthetics to provide prolonged analgesia from a single injection has encountered three principal challenges: inadequate duration of action, systemic toxicity, and adverse local tissue reaction. A wide variety of controlled-release technologies has been employed to extend the duration of nerve block, but most such systems result at best in a several-fold extension of duration compared to unencapsulated drugs. Approaches that encapsulate synergistic drug combinations have achieved nerve blocks lasting many days. For example, co-encapsulation of bupivacaine and dexamethasone in polymeric microspheres produced nerve blocks lasting more than four days (Drager, et al., Anesthesiology, 89(4):969-979 (1998). Co-encapsulation of site 1 sodium channel blockers (which block the sodium channel at site 1 on the outer surface) with conventional local anesthetics also greatly prolonged sciatic nerve blockade. Addition of dexamethasone prolonged the sciatic nerve blockade to more than nine days in the rat (Kohane, et al, Pain, 104(1-2):415-421 (2003). However, tissue reaction to such formulations has been problematic. Conventional local anesthetics are intrinsically myotoxic (Padera, et al., Anesthesiology, 108(5):921-928 (2008); Pere, et al., Reg Anesth, 18(5):304-307 (1993)). They are also myotoxic when released from a wide range of delivery systems (Padera, et al., Anesthesiology, 108(5):921-928 (2008); Jia, et al., Biomaterials, 25(19):4797-4804 (2004)), even when the delivery systems themselves are minimally toxic. The myotoxicity of bupivacaine increases dramatically over extended durations of exposure (Padera, et al., Anesthesiology, 108(5):921-928 (2008)), suggesting that myotoxicity may be an inevitable consequence of sustained release of such compounds. Conventional local anesthetics are also neurotoxic (Zimmer, et al., Anaesthesist, 56(5):449-453 (2007); Yamashita, et al., Anesth Analg, 97(2):512-519 (2003)). The presence of particles themselves enhances local anesthetic myotoxicity in viva (Padera, et al., Anesthesiology, 108(5):921-928 (2008), and can cause inflammatory responses at the nerve that may considerably outlast the duration of blockade (Kohane, et al, Pain, 104(1-2):415-421 (2003); Padera, et al., Anesthesiology, 108(5):921-928 (2008); and Kohane, et al., J Biomed Mater Res., 59(3):450-459 (2002)).

U.S. Pat. No. 6,326,020 to Kohane, et al. discloses compositions containing a combination of naturally occurring site 1 sodium channel blockers, with other agents such as local anesthetics, vasoconstrictors, glutocorticoids, or adrenergic drugs for prolonged duration of nerve block. Site 1 sodium channel blockers do not cause myo- or neurotoxicity (Barnet, et al., Pain 110(1-2):432-438 (2004); Sakura, et al., Anesth Analg, 81(2):338-346 (1995)), which makes them desirable for an extended release formulation. U.S. Pat. No. 6,326,020 discloses poly(lactic acid-glycolic acid) microspheres containing TTX (at 0.1% theoretical loading) alone, in an carrier fluid containing epinephrine, which produces nerve block lasting about six hours with an onset of more than one hour. TTX without epinephrine has been shown to produce sciatic nerve block, but with considerable toxicity at the most effective doses (Kohane, et al., Anesthesiology, 89(1):119-131 (1998). Studies by Kohane, et al, Pain, 104(1-2):415-421 (2003) employing polymeric microspheres of TTX alone showed that TTX was lethal (at 0.1% w/w) or ineffective for nerve block (at 0.05% w/w), producing a median block of 0 min. Additionally, it is extremely difficult to encapsulate effectively these extremely potent local anesthetics in polymeric particles since they are hydrophilic and the systemic toxicity from their initial rapid release is dose-limiting (Barnet, et al., Anesth Analg, 101(6):1838-1843 (2005); Kohane, et al., Anesthesiology, 89(1):119-131 (1998)). This makes the development of particulate systems based entirely on such compounds (i.e. without inclusion of conventional local anesthetics) very difficult.

There is still a need for a composition containing a site 1 sodium channel blocker that can provide a reliable prolonged nerve block while avoiding local tissue toxicity without the need of a local anesthetic.

It is an object of the present invention to provide a composition containing a site 1 sodium channel blocker for use as an anesthetic with increased potency, efficacy and rapid onset.

It is still another object of the present invention to provide a method for local anesthesia that avoids systemic and local tissue toxicity due to the local anesthetic and provides rapid onset of nerve block, which is also prolonged, without those detrimental sequelae.

### SUMMARY OF THE INVENTION

Compositions containing site 1 sodium channel blockers for use as local anesthetics with rapid nerve block, improved potency and efficacy, and no local toxicity have been developed. Liposomes were employed for increased loading of the site 1 sodium channel blocker, producing prolonged duration of block without systemic toxicity. In one embodiment, the compositions contain a site 1 sodium channel blocker alone. In another embodiment, the compositions contain a site 1 sodium channel blacker in combination with a corticosteroid. In a preferred embodiment, the Site I sodium channel blacker is saxitoxin. In another preferred embodiment, when the site 1 sodium channel blocker is combined with a corticosteroid, the preferred corticosteroid is dexamethasone.

As demonstrated by the examples, encapsulating site 1 sodium channel blockers in liposomes results in rapid and prolonged nerve block without systemic toxicity, which is enhanced by the addition of a corticosteroid. Fluid liposomes showed more rapid release of STX than did solid ones. Dexamethasone accelerated STX release.

### BRIEF DESCRIPTION OF THE DRAWINGS

FIGS. 1A, 1B and 1C are graphs showing the cumulative release over time in hours of total encapsulated bupivacaine (1A) and saxitoxin (1B) from liposomes in vitro. FIG. 1C is a graph showing release of encapsulated compounds (Dexamethasone) over time from liposome formulations in vitro expressed as a cumulative percentage of total encapsulated drug. Data are means with standard deviations (n=4). FIGS. 1A-1C provide release profiles for the following formulations: free bupivacaine (FIG. 1A) or free saxitoxin (STX, FIG. 1B) (-x-); fluid liposomes loaded with a mixture of bupivacaine and STX (-▪-); fluid liposomes loaded with a mixture of bupivacaine, STX, and dexamethasone (1 mg/mL) (-♦-) and (5 mg/mL) (--); solid liposomes loaded with a mixture of bupivacaine and STX (-□-); and solid liposomes loaded with a mixture of bupivacaine, STX, and dexamethasone (1 mg/mL) (-⋄-) and (5 mg/mL) (-◯-). FIG. 1B also refers to release profiles for fluid STX (-) and solid liposomes loaded with STX (-Δ-).

FIGS. 2A and 2B are graphs showing percentage cell survival after exposure to different dosages (mg/ml) of free bupivacaine in C2C12 and PC12 cells, respectively. Exposure time in FIG. 2A was 2 hrs (-▪-), 6 hrs (-□-), 24 hrs (--), 48 hrs (-▴-), and four days (-♦-). Exposure time in FIG. 2B additionally includes 1 day (-▴-) and seven days (-x-).

FIGS. 2C and 2D are graphs showing percentage of cell survival after exposure to different dosages (mg/ml) of free saxitoxin (STX) in C2C12 and PC12 cells, respectively. In C2C12 cells (FIG. 2C), survival was measured with exposure times of 2 hrs (-Δ-), 6 hrs (-▪-), 24 hrs (--), 48 hrs (-□-), and 4 days (-◯-). In PC12 cells (FIG. 2D), survival was measured with exposure times of 6 hours (-▪-), 24 hours (-▴-), and 7 days (--).

FIGS. 2E and 2F are graphs showing percentage of cell survival after exposure to different dosages (mg/ml) of fluid liposomes encapsulating combinations of bupivacaine, dexamethasone and/or saxitoxin in C2C12 and PC12 cells, respectively. The following formulations were tested in C2C12 cells (FIG. 2E), free bupivacaine (-♦-); fluid liposomes loaded with bupivacaine (-▪-); fluid liposomes loaded with a mixture of bupivacaine and STX (-◯-); fluid liposomes loaded with a mixture of bupivacaine and dexamethasone (5 mg/ml) (-Δ-); and fluid liposomes loaded with a mixture of bupivacaine, STX, and dexamethasone (5 mg/ml) (-▪-). The following formulations were tested in PC12 cells (FIG. 2F), free bupivacaine (-◯-); fluid liposomes loaded with bupivacaine (-▪-); fluid liposomes loaded with a mixture of bupivacaine and STX (--); fluid liposomes loaded with a mixture of bupivacaine and dexamethasone (5 mg/ml) (-▴-). In 2E and 2F, data for free bupivacaine are reproduced from 2A and 2B for comparison. Data are means with standard deviations (n=4).

FIG. 3 is a graph showing the duration of sensory and motor block in hours in animals injected with liposomes containing STX and/or bupivacaine and dexamethasone. Data are means with standard deviations (n=8). The dotted line denotes identical durations of sensory and motor block. From lower left to upper right, the data points on the dotted line correspond to: a mixture of fluid and solid liposomes loaded with bupivacaine; fluid liposomes loaded with STX; fluid liposomes loaded with bupivacaine and STX; solid liposomes loaded with STX and dexamethasone (5 mg/ml); solid liposomes loaded with STX; solid liposomes loaded with bupivacaine and STX; and solid liposomes loaded with STX and dexamethasone (0.8 mg/ml).

FIG. 4 shows Real Time PCR of mRNA from dorsal root ganglia showing real time gene expression from animals injected with liposomes (empty or containing test compounds), or a toxic concentration of amitriptyline. n=3 for each treatment. Asterisk denotes p<0.001, in the comparison of amitriptyline treatment to all liposomal treatments.

### DETAILED DESCRIPTION OF THE INVENTION

### I. Definitions

As used herein a site 1 sodium channel blocker is a molecule that binds the outer opening of sodium channels at a location termed “site 1”. In a preferred embodiment, the site 1 sodium channel blocker is a naturally occurring toxin or a derivative thereof.

The term “derivative thereof” as used herein includes any derivative of a site 1 sodium channel blocker having substantially the same functional properties as the non-derivatized site 1 sodium channel blocker such as biological and/or pharmacological, i.e. to effectively block sodium channels.

### II. Compositions

The composition is designed to prolong the duration of a local anesthetic block, with no systemic toxicity. The composition consists of a Site 1 sodium channel blocker alone or in combination with a corticosteroid, administered in a pharmaceutically acceptable carrier such as a liposome in amounts effective to prolong the duration of block of the local anesthetic, with no systemic toxicity. The composition is administered in a formulation locally at the site where the nerve is to be blocked, preferably as a suspension.

A. Site 1 Sodium Channel Blockers

Site I sodium channel blockers include tetrodotoxin (TTX), saxitoxin (STX), decarbamoyl saxitoxin, neosaxitoxin, and the gonyautoxins (referred to jointly herein as “toxins”). The preferred toxins are TTX and STX.

Tetrodotoxins are obtained from the ovaries and eggs of several species of puffer fish and certain species of California newts. Chemically, it is an amino perhydroquinoline. See Kao, Pharmacological Reviews, 18(2):997-1049 (1966). Tetrodotoxin alone is too toxic to be used as an anesthetic.

Saxitoxin was first extracted from the Alaska butterclam, Saxidomus gigantcus, where it is present in algae of the genus Gonyaulax. The reported chemical formula is C₁₀H₁₅N₇O₃.2HCl. It is believed the toxin has a perhydropurine nucleus in which are incorporated two guanidinium moieties. Saxitoxin is too toxic to be used alone as a local anesthetic.

A number of polypeptides have been isolated from the paralytic venoms of the fish hunting cone snails of the genus Conus found in the Philippine archipelago. Designated “conotoxins,”, these have been discovered to affect ion channel function. The paralytic a, m, and w conotoxins block nicotinic acetylcholine receptors, sodium channels, and voltage sensitive calcium channels, respectively (reviewed in Olivera, et al., Science, 249:257-263 (1990)). Those which block sodium channels can be used in the same manner as the tetrodotoxins and saxitoxins.

Dosage ranges are between 28 and 2800 micrograms, with a loading in the liposomes of between 0.1 to 90% by weight, more preferably between 5 and 75%.

B. Corticosteroids

Corticosteroids that are useful to prolong in vivo nerve blockade include glucocorticoids such as dexamethasone, cortisone, hydrocortisone, prednisone, and others routinely administered orally or by injection. Other glucocorticoids include beclomethasone, betamethasone, flunisolide, methyl prednisone, para methasone, prednisolone, triamcinolome, alclometasone, amcinonide, clobetasol, fludrocortisone, difluorosone diacetate, fluocinolone acetonide, fluoromethalone, flurandrenolide, halcinonide, medrysone, and mometasone, and pharmaceutically acceptable salts and mixtures thereof. The relative strengths of the different corticosteriods are well known, and described, for example, in Goodman and Gilman's.

Typically these are administered at between 0.05 and 1 mg dexamethasone/mg, or equivalent based on strength of glucocorticoid (weaker requiring more, stronger requiring less).

C. Carriers

In the preferred embodiment, the carrier is a liposome, stored in a vial as a dry powder, or suspended in an aqueous solution for injection. Liposomes (LPs) are spherical vesicles, composed of concentric phospholipid bilayers separated by aqueous compartments. LPs have the characteristics of adhesion to and creating a molecular film on cellular surfaces. Liposomes are lipid vesicles composed of concentric phospholipid bilayers which enclose an aqueous interior (Gregoriadis, et al., Int J Pharm 300, 125-30 2005 (2005); Gregoriadis and Ryman, Biochem J 124, 58P (1971)). The lipid vesicles comprise either one or several aqueous compartments delineated by either one (unilamellar) or several (multilamellar) phospholipid bilayers (Sapra, et al., Curr Drug Deliv 2, 369-81 (2005)). The success of liposomes in the clinic has been attributed to the nontoxic nature of the lipids used in their formulation. Both the lipid bilayer and the aqueous interior core of liposomes can serve the purpose of treatment. Liposomes have been well studied as carrier of toxins for enhancing their efficacy at lower doses (Alam, et al., Mol Cell Biochem 112, 97-107 1992; Chaim-Matyas, et al., Biotechnol Appl Biochem 17 (Pt 1), 31-6 1993; de Paiva and Dolly, FEBS Lett 277, 171-4 (1990); Freitas and Frezard, Toxicon 35, 91-100 (1997); Mandal and Lee, Biochim Biophys Acta 1563, 7-17 (2002)).

Liposomes have been widely studied as drug carriers for a variety of chemotherapeutic agents (approximately 25,000 scientific articles have been published on the subject) (Gregoriadis, N Engl J Med 295, 765-70 (1976); Gregoriadis, et al., Int J Pharm 300, 125-30 (2005)). Water-soluble anticancer substances such as doxorubicin can be protected inside the aqueous compartment(s) of liposomes delimited by the phospholipid bilayer(s), whereas fat-soluble substances such as amphotericin and capsaicin can be integrated into the phospholipid bilayer (About-Fadl, Curr Med Chem 12, 2193-214 (2005); Tyagi, et al., J Urol 171, 483-9 (2004)). Delivery of chemotherapeutic agents leads to improved pharmacokinetics and reduced toxicity profile (Gregoriadis, Trends Biotechnol 13, 527-37 (1995); Gregoriadis and Allison, FEBS Lett 45, 71-4 1974; Sapra, et al., Curr Drug Deliv 2, 369-81 (2005)). More than ten liposomal and lipid-based formulations have been approved by regulatory authorities and many liposomal drugs are in preclinical development or in clinical trials (Barnes, Expert Opin Pharmacother 7, 607-15 (2006); Minko, et al., Anticancer Agents Med Chem 6, 537-52 (2006)). Fraser, et al. Urology, 2003; 61: 656-663 demonstrated that intravesical instillation of liposomes enhanced the barrier properties of dysfunctional urothelium and partially reversed the high micturition frequency in a rat model of hyperactive bladder induced by breaching the uroepithelium with protamine sulfate and thereafter irritating the bladder with KCl. Tyagi et al. J Urol., 2004; 171; 483-489 reported that liposomes are a superior vehicle for the intravesical administration of capsaicin with less vehicle induced inflammation in comparison with 30% ethanol. The safety data with respect to acute, subchronic, and chronic toxicity of liposomes has been assimilated from the vast clinical experience of using liposomes in the clinic for thousands of patients. The safe use of liposomes for the intended clinical route is also supported by its widespread use as a vehicle for anticancer drugs in patients.

Liposomes have previously been used for controlled release of conventional anesthetics (Grant, et al., Anesthesiology, 101(1):133-137 (2004); Grant, et al., Clin Exp Pharmacol Physiol., 30(12):966-968 (2003); and Malinovsky, et al., J Control Release., 60(1):111-119 (1999)). Bupivacaine liposomes provide skin analgesia lasting 14-29 hr in a mouse model (Grant, et al., Pharm Res., 18(3):336-343 (2001)), and 42 h in humans Grant, et al., Anesthesiology, 101(1):133-137 (2004). Liposomal formulations of various local anesthetics have been used in rat infraorbital nerve blockade, with durations of 65-110 min (Cereda., et al., Can J Anaesth., 53(11):1092-1097 (2006); de Araujo., et al., Can J Anaesth., 51(6):566-572 (2004)).

Preferred phospholipids are the naturally occurring phospholipids such as 1,2 dimyristoylsn-glycero-3-phosphocholine (DMPC), 1,2-distearoyl-snglycero-3 phosphocholine (DSPC), 1,2-distearoyl-sn-glycero-3-phosphatidylglycerol, sodium salt (DSPG), and 1,2-dimyristoylsn-glycero-3-phosphoglycerol, sodium salt- (DMPG). In the preferred embodiment, liposomes were produced with DMPC and DMPG, or DSPC and DSPG (DMPC=1,2-dimyristoyl-sn-glycero-3-phosphocholine, DMPG=2-dimyristoyl-sn-glycero-3-phosphoglycerol, DSPC=1,2-distearoyl-sn-glycero-3-phosphocholine, DSPG=1,2-distearoyl-sn-glycero-3-phosphatidylglycerol), Those made with DMPC are referred to as “fluid” liposomes; those with DSPC as “solid” based on their phase transition temperatures (Tₘ). Particles were loaded with bupivacaine, STX, and/or dexamethasone.

The compositions can be provided in any pharmaceutically acceptable carrier for injection, such as water, saline, dextrose solutions, carboxymethylcellulose, mannitol, and buffered solutions.

### III. Methods of Administration

The composition can be administered by any of the methods for administering local anesthetics known to one of ordinary skill in the art. The composition can be formulated for topical anesthesia, infiltration anesthesia, filed block anesthesia, nerve block anesthesia, intravenous regional anesthesia, spinal anesthesia and epidural anesthesia.

The present invention will be further understood by reference to the following non-limiting examples.

### IV. Examples

### Example 1

### Comparison of Polymeric Microparticle and Liposomal Encapsulated STX for Efficacy, Duration of Block and Toxicity

Materials and Methods

Materials. Saxitoxin (STX) stock solution was supplied by the U.S. Food and Drug Administration. Acetonitrile, ammonium sulfate, bupivacaine hydrochloride, chloroform, HPLC grade dexamethasone, sodium chloride, methanol, and octyl-D-glucopyranoside (OGP) were from Sigma; 1,2 dimyristoylsn-glycero-3-phosphocholine (DMPC), 1,2-distearoyl-snglycero-3 phosphocholine (DSPC), 1,2-distearoyl-sn-glycero-3-phosphatidylglycerol, sodium salt (DSPG), and 1,2-dimyristoylsn-glycero-3-phosphoglycerol, sodium salt- (DMPG) were purchased from Genzyme. Tert-butanol was purchased from Riedel-de Haen.

Liposome Preparation

Liposomes were prepared by modified thin lipid film hydration (Szoka, et al., Annual review of biophysics and bioengineering, 9:467-508 (1980). Lipids were selected to produce relatively fluid (DMPC-DMPG) or solid (DSPC-DSPG) liposomes at 37° C., (phase transition temperatures, Tₘ; DSPC=56° C. and DMPC=23° C.). DSPC:DSPG:cholesterol or DMPC:DMPG:cholesterol (molar ratio 3:1:2) were dissolved in t-butanol. Dexamethasone was added in some samples prior to lyophilization. The lyophilized cake was hydrated with 250 mM ammonium sulfate or, in some groups, with 0.1 mg STX, at 55-60° C. The suspension was homogenized at 10,000 rpm with a ⅜″ Mini-Micro workhead on a L4RT-A Silverson Laboratory Mixer (East Longmeadow, Mass.) for 10 minutes followed by ten freeze-thaw cycles. Excess free STX was removed by centrifugation (4000 rpm, 4° C. for 20 minutes) and replaced by 2 mL of 30 mg/mL bupivacaine hydrochloride in 20 mM citrate buffer pH 4.0, or with 0.9% saline if bupivacaine was not desired. Liposome suspensions with bupivacaine added were stirred at 50° C. for 4-6 hours. Liposome suspensions were dialyzed against 0.9% saline solution in 50 kDa molecular weight cut-off dialysis bags for 48 hours. Drug-free liposomes were prepared by the same procedure, omitting the drug.

Liposome Characterization

Liposomes were sized with a Beckmann Coulter Counter Multisizer 3 (Fullerton, Calif.). Zeta potentials were measured using Brookhaven Instruments Corporation ZetaPALS and ZetaPlus software (Holtsville, N.Y.). Liposome drug concentrations were determined following disruption of the liposomes with octyl β-D-glucopyranoside (OGP). Dexamethasone and bupivacaine were quantitated by HPLC (Agilent HPLC 1100 Series system, Canada) at 254 and 215 nm, respectively, using methods from the United States Pharmacopeia. Determination of STX concentration was based on the method of Bates, Kostriken and Rapoport (Bates, et al., Journal of agricultural and food chemistry., 26(1):252-254 (1978)) in which saxitoxin is oxidized to fluorescent products. Lipid concentrations were determined by colorimetry by the Bartlett assay (Bartlett, J. Biol. Chem., 234(3):466-468 (1959)).

To assess drug content, liposomes were first destroyed by adding them in a 1:2 ratio to 100 mMOGP. The resulting solution was then analyzed as described below. STX concentration determination was based on the method of Bates et al. (Bates, et al., Journal of agricultural and food chemistry., 26(1):252-254 (1978)). Samples, standards, or blanks (0.3 mL) and 30% hydrogen peroxide (0.05 mL) were mixed vigorously with 5.0 mL 1.0M NaOH and 4.7 mL Milli-Q water. After 40 min at room temperature, the mixture received 0.7 mL concentrated acetic acid. Fluorescence was measured in a 1 cm cuvette in a PerkinElmer LS-50B, 330-nm excitation, 380-nm emission, excitation and emission slits 10 nm. The concentrations of DSPC, DSPG, DMPC, and DMPG were determined colorimetrically by the Bartlett assay (Bartlett, J. Biol. Chem., 234(3):466-468 (1959)), which assessed the amount of phosphorus after hydrolysis of the phospholipids, with 1 mole of phosphorus equivalent to 1 mole of phospholipids. Samples, standards or blanks (0.2 mL) were mixed in 0.4 mL 10N H2SO4, and heated to 175° C. for 1 h. Subsequently, 0.03 mL of 30% hydrogen peroxide was added, and samples heated to 175° C. for 1 h; 2.3 mL of 22 mM ammonium molybdate, 2.3 mL of 0.44 mM H2SO4 and 0.2 mL of 0.1 mM 1-amino-2-naphthol-4-sulfonic acid (ANSA; Sigma) were added, and samples were boiled at 100° C. for 7 min. Absorbance was measured at 830 nm.

In Vitro Drug Release

One mL of liposomes or compounds in solution were inserted into the lumen of a SpectraPor 1.1 Biotec Dispodialyzer (Spectrum Laboratories, Rancho Domingeuz, Calif.) with a 25,000 MW cut-off. The dialysis bag was placed in a test tube with 12 mL phosphate buffered saline and incubated at 37° C. on a tilt-table (Ames Aliquot, Miles). At predetermined intervals, the dialysis bag was transferred to a new test tube with fresh phosphate buffered saline that was pre-warmed to 37° C. Concentrations of compounds were quantitated as above.

Stability

Stability was determined by examining changes in vesicle size, zeta potential, liposome integrity, and drug and lipid leakage (disruption of the membrane) over time at room temperature (21° C.) and 4° C. At specific time points, 400 μl of the liposomal formulation was centrifuged with a Centricon separation filter (30,000 MW Millipore, Billerica, Mass.) at 3500 g, for 30 min, at 4° C. The liposomes were retained in the upper chamber. 100-150 μl of the filtrate was recovered from the lower chamber, in which drug and lipid concentrations were determined. Leakage, liposome integrity, size distribution and zeta potential were evaluated every day for two weeks.

Cell Culture

C2C12 mouse myoblasts (American Type Culture Collection (ATCC) CRL-1772, Manassas, Va.) were cultured to proliferate in Dulbecco's modified Eagles medium (DMEM) supplemented with 20% fetal bovine serum (FBS) and 1% Penicillin Streptomycin. Cell culture supplies were obtained from Invitrogen (Carlsbad, Calif.) unless otherwise noted. Cells were plated at 50,000 cells/mL in DMEM with 2% horse serum and 1% Penn Strep, and left to differentiate into myotubules for 10-14 days. During differentiation, media were exchanged every 2 to 3 days. Cell viability and proliferation were studied after exposures to liposomes, free drugs, and empty liposomes with free drug for up to 96 hr (see below).

PC12 cells (ATCC, CRL-1721) originating from rat adrenal gland pheochromocytoma were grown in 24-well tissue culture dishes (CellBind, Corning N.Y.) with F-12K (ATCC) supplemented with 12.5% horse serum (Gibco, Carlsbad, Calif.), 2.5% fetal bovine serum (Gibco), and 1% Penn Strep (Sigma, St. Louis, Mo., USA). For neuronal induction (PC12), cells were seeded at a relative low density of 5×10⁴ cells/cm² and 50 ng/mL nerve growth factor (NGF) was added 24 hr after seeding. Cell viability and proliferation were evaluated as for C2C12 cells. Experiments with PC12 cells were conducted for up to 7 days.

Cell Viability Assay

Cell viability was assessed after adding drug- or particle-containing media by a colorimetric assay (MTT kit, Promega G4100 Madison, Wis.) at selected time points. At each time point 150 μL of 3-(4,5-dimethylthiazol-2-yl)-2,5-diphenyl tetrazolium bromide was added, and then cells were incubated at 37° C. for 4 h, then 1 mL solubilization solution (detergent) was added. Absorbance was read at 570 nm with a SpectraMax 384 Plus fluorometer (Molecular Devices) after samples were incubated in the dark overnight. Cells were also monitored visually to confirm the results of the assay. Each plate had wells that contained media without cells or other additives whose absorbance was subtracted from the rest of the plate as background. All groups were then normalized to those wells.

Sciatic Nerve Block and Neurobehavioral Testing

Animals were cared for in compliance with protocols approved by the Children's Hospital Animal Care and Use Committee, and the Massachusetts Institute of Technology Committee on Animal Care, which conformed to guidelines of the International Association for the Study of Pain (Zimmermann, Pain, 16(2):109-110 (1983)). Adult male Sprague-Dawley rats (Charles River Laboratories, Wilmington, Mass.) weighing 320-420 g were housed in groups, in a 6 AM -6 PM light-dark cycle. Under brief isoflurane-oxygen anesthesia, a 23G needle was introduced postero-medial to the greater trochanter, until bone was contacted, and 0.6 mL of test solution was injected over the sciatic nerve. Thermal nociception was assessed by a modified hotplate test (Padera, et al., Anesthesiology, 108(5):921-928 (2008); Pere, et al., Reg Anesth., 18(5):304-307 (1993)), and motor function via a weight-bearing test (Kohane, et al., Anesthesiology, 89(1):119-131 (1998); Thalhammer, et al., Anesthesiology, 82(4):1013-1025 (1995)).

In brief, hind paws were exposed in sequence (left then right) to a 56° C. hot plate (Model 39D Hot Plate Analgesia Meter; IITC). The time (latency) until paw withdrawal was measured by a stopwatch. Thermal latency in the uninjected leg was a control for systemic effects of the injected agents. If the animal did not remove its paw from the hot plate within 12 s, it was removed by the experimenter to avoid injury to the animal or the development of hyperalgesia. The experimenter was blinded as to what treatment specific rats were receiving. The duration of thermal nociceptive block was calculated as the time required for thermal latency to return to a value of 7 s from a higher value; 7 s is the midpoint between a baseline thermal latency of ˜2 seconds in adult rats, and a maximal latency of 12 s. Motor strength was assessed with a weight bearing test. In brief, the animal was held over a digital balance such that it could bear weight with one hind paw at a time. The maximum weight that it could bear was recorded. The duration of motor blockade was defined as the time for weight bearing to return halfway to normal from maximal block. The halfway point for each rat was defined as [(highest weight borne by either leg)−(lowest weight borne by blocked leg)]/2+(lowest weight borne by blocked leg).

Necropsy and Histological Analysis.

Rats were euthanized by carbon dioxide at 4, 14 and 21 days. The nerve and surrounding tissues were harvested and histological hematoxylin-eosin sections were produced with standard techniques. Samples for Epon-embedded sections were fixed for 24 hrs at 24° C. in Karnovsky's KII Solution (2.5% glutaraldehyde, 2.0% paraformaldehyde, 0.025% calcium chloride in 0.1M sodium cacodylate buffer [Aldrich, St. Louis, Mo.] pH 7.4). Samples were post-fixed in osmium tetroxide, stained with uranyl acetate, dehydrated in graded ethanol solutions, and infiltrated with propylene oxide/Epon mixtures. Subsequently, 1 μm sections were cut on an ultramicrotome and stained with toluidine blue. Photomicrographs were obtained using a Nikon Eclipse 50i microscope (Melville, N.Y.) with SPOT Insight 4 Meg FW Color Mosaic camera and SPOT 4.5.9.1 software from Diagnostic Instruments, Inc. (Sterling Heights, Mich.).

Gene Expression

RNA Isolation

The L4 and L5 dorsal root ganglia were removed on necropsy and immediately frozen in liquid nitrogen. Tissue samples were stored at −80° C. until use. Total RNA was extracted from homogenized DRG samples by acid phenol extraction (TRIzol reagent; Gibco-BRL, CA), and isolated with a Qiagen RNeasy Mini kit column (QIAGEN, CA). The purity and concentration of RNA samples were determined from the absorbencies at 260 and 280 nm, with a NanoDrop 100 spectrophotometer (NanoDrop Technologies, Wilmington, Del.).

Real Time PCR

Total DRG RNA samples underwent reverse transcription with SuperScript III (Invitrogen) following the manufacturer's procedure. Real-time PCR reactions for each sample were run in duplicate using 100 ng of cDNA in Taqman gene expression assays (Applied Biosystems) according to the manufacturer's instructions. Real time PCR was performed using Applied Biosystems' Step One equipment and program. The relative amount of specifically amplified cDNA was calculated using the delta-CT method (Vandesompele, et al., Genome biology, 3(7):RESEARCH0034-1-0034.11 (2002); Hoebeeck, et al., Laboratory investigation; A Journal of Technical Methods and Pathology, 85(1):24-33 (2005)). The Applied Biosystems primers used are as follows: GAPDH: Rn99999916_S1, β-actin: Rn00667869_m1; Gadd45 α: Rn00577049_m1; ATF3: Rn00563784_m1; Cacna2d1: Rn01442580_m1; Smagp: Rn00788145_g1.

Statistics

Data are presented as means±standard deviations (n=4 in release kinetics, cell work, and gene expression; n=8 in neurobehavioral studies). To take multiple comparisons into account, all statistical comparisons were done with the Tukey-Kramer test, using InStat software (GraphPad, San Diego Calif.). A P-value <0.05 was considered to denote statistical significance.

Results

Liposomal Formulations

Liposomes were produced with DMPC and DMPG, or DSPC and DSPG (DMPC=1,2-dimyristoyl-sn-glycero-3-phosphocholine, DMPG=2-dimyristoyl-sn-glycero-3-phosphoglycerol, DSPC=1,2-distearoyl-sn-glycero-3-phosphocholine, DSPG=1,2-distearoyl-sn-glycero-3-phosphatidylglycerol) (TABLE 1). Those made with DMPC are referred to as “fluid” liposomes; those with DSPC as “solid” based on their phase transition temperatures (Tₘ). Particles were loaded with bupivacaine, STX, and/or dexamethasone.

TABLE 1
Characterization of Liposomes

| | Compound concentration, mg/mL | | | | |
|---|---|---|---|---|---|
| Lipid | | | | | Zeta |
| composition | Bupivacaine | Dexamethasone | STX | Size, μm⁺ | potential, mV |
| DSPC, | 9.93 ± o.54 | — | — | 4.0 ± 1.5 | −32.2 ± 2.2 |
| “solid”* | 10.22 ± 0.71 | 3.96 ± 0.11 | — | 4.1 ± 1.3 | −30.2 ± 2.1 |
| | — | — | 0.031 ± 0.001 | 4.0 ± 1.2 | −33.9 ± 2.1 |
| | — | 4.61 ± 0.23 | 0.027 ± 0.001 | 4.0 ± 1.6 | −32.8 ± 2.2 |
| | — | 1.13 ± 0.24 | 0.029 ± 0.001 | 4.0 ± 1.4 | −33.7 ± 2.1 |
| | — | 0.81 ± 0.03 | 0.030 ± 0.001 | 4.0 ± 1.2 | −32.8 ± 3.2 |
| | — | 0.31 ± 0.01 | 0.030 ± 0.001 | 4.0 ± 1.3 | −32.3 ± 2.4 |
| | 9.02 ± 0.2 | — | 0.022 ± 0.0005 | 3.8 ± 1.2 | −36.0 ± 2.1 |
| | 9.91 ± 0.1 | 4.34 ± 0.06 | 0.021 ± 0.0004 | 3.9 ± 1.2 | −35.6 ± 2.3 |
| | 8.86 ± 0.12 | 1.01 ± 0.035 | 0.022 ± 0.0001 | 4.0 ± 1.3 | −34.6 ± 2.1 |
| DMPC, | 9.28 ± 1.1 | — | — | 4.2 ± 1.5 | −33.6 ± 2.0 |
| “fluid”* | 8.74 ± 0.98 | 4.56 ± 0.76 | — | 3.9 ± 1.4 | −33.6 ± 2.5 |
| | — | — | 0.030 ± 0.001 | 4.3 ± 1.0 | −32.0 ± 2.3 |
| | — | 4.51 ± 0.97 | 0.026 ± 0.001 | 4.1 ± 1.8 | −36.4 ± 2.4 |
| | — | 0.98 ± 0.02 | 0.028 ± 0.001 | 4.2 ± 1.3 | −35.6 ± 2.0 |
| | — | 0.65 ± 0.01 | 0.029 ± 0.001 | 4.2 ± 1.1 | −35.5 ± 2.1 |
| | — | 0.28 ± 0.01 | 0.030 ± 0.001 | 4.3 ± 1.0 | −36.0 ± 2.0 |
| | 8.95 ± 0.87 | — | 0.019 ± 0.001 | 4.0 ± 1.1 | −37.5 ± 1.2 |
| | 9.12 ± 1.21 | 4.60 ± 0.3 | 0.018 ± 0.002 | 3.8 ± 1.2 | −33.0 ± 1.3 |
| | 8.84 ± 1.03 | 1.1 ± 0.04 | 0.019 ± 0.001 | 3.9 ± 1.0 | −32.0 ± 1.1 |

Data are means ± SD, n = 4.
*The labels by which particles with these lipid compositions are referred to in the text.
±Median volume weighted diameter.

The median volume-weighted diameters of both fluid and solid liposomes were approximately 4.0 μm, with median zeta potentials of approximately −35 mV, irrespective of drug content (TABLE 1). The mean encapsulation efficiencies of bupivacaine in solid and fluid liposomes were 64 and 60%, respectively. The liposomal drug loadings, in mg/mL, were 8.7-10.2 for bupivacaine (i.e. approximately 1% w/v), 0.03 to 0.018 for saxitoxin, and 50-60 for lipids.

In Vitro Drug Release

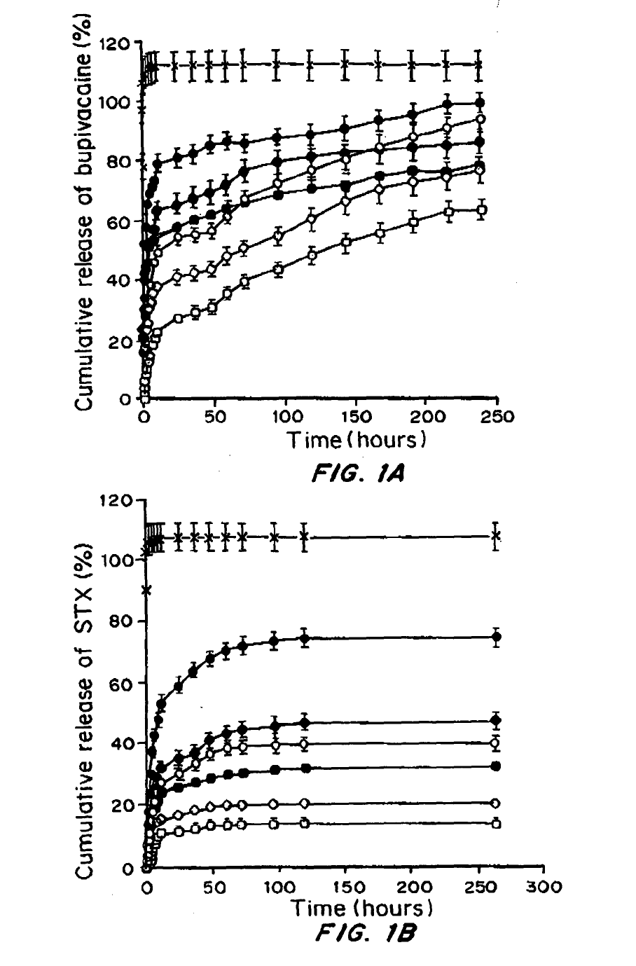

Release kinetic studies were performed at 37° C. To assess the potential of these liposomes to provide sustained nerve blockade. All liposome formulations significantly increased the duration of bupivacaine release compared to free bupivacaine (e.g. p<0.001 at 50 hours; FIG. 1A). Fluid particles showed faster (e.g. p<0.05 at 50 hours) release than solid ones. Release of bupivacaine was increased in proportion to the amount of dexamethasone incorporated (e.g. p<0.05 when comparing 0 to 5 mg/mL dexamethasone at 50 hours). Dexamethasone release was not affected by lipid type (FIG. 1C). However, particles with a higher proportion of dexamethasone (5 mg/mL) showed slower release of dexamethasone on a percentage basis, particularly at later time points (e.g. p<0.05 when comparing 1 to 5 mg/mL dexamethasone at 50 hours). Fluid liposomes showed more rapid release of STX (FIG. 1B) than did solid ones (p<0.05 at all time points, and <0.001 after 50 hours), and dexamethasone accelerated STX release (p<0.05). These results, which showed sustained release of the compounds of interest for several days, supported their potential to provide prolonged duration local anesthesia.

Stability

At 4° C. both solid and fluid liposomes (containing all compounds, singly and in combination) were stable for two weeks: drug leakage was less than 3% over that period, and there was no significant change in liposome size, zeta potential and drug to lipid ratio. At 21° C. particles made with both lipid compositions showed more than 10% release of bupivacaine in 48 hr, i.e. they were not stable. Release of saxitoxin and dexamethasone were slower than for bupivacaine, but still higher than at 4° C.

Cytotoxicity

One of the underlying hypotheses of this work was that prolonged exposure to saxitoxin would cause less injury to muscle and nerve than bupivacaine.

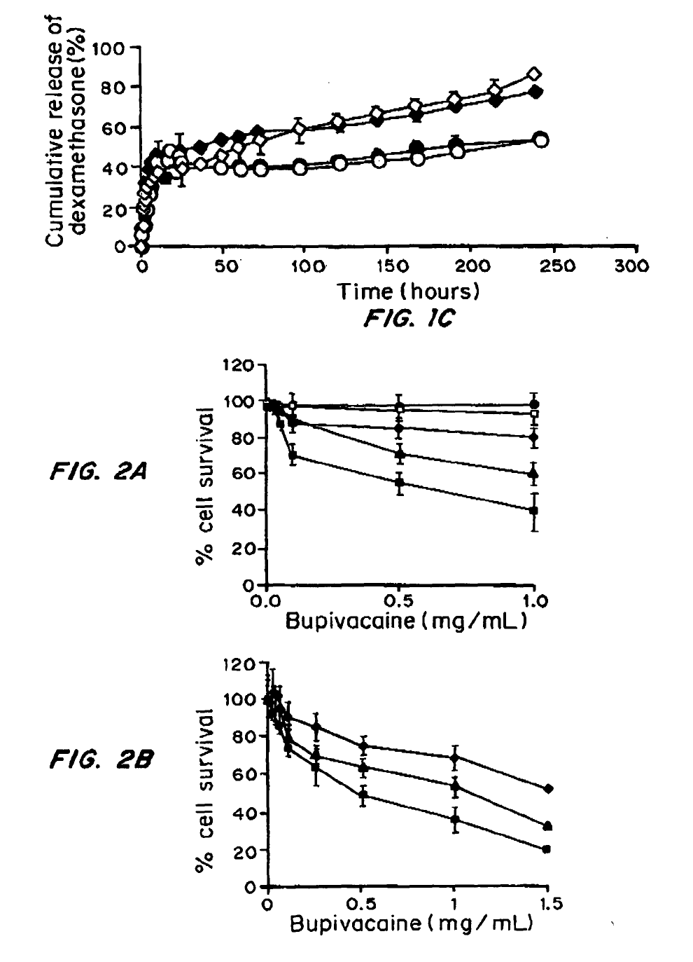

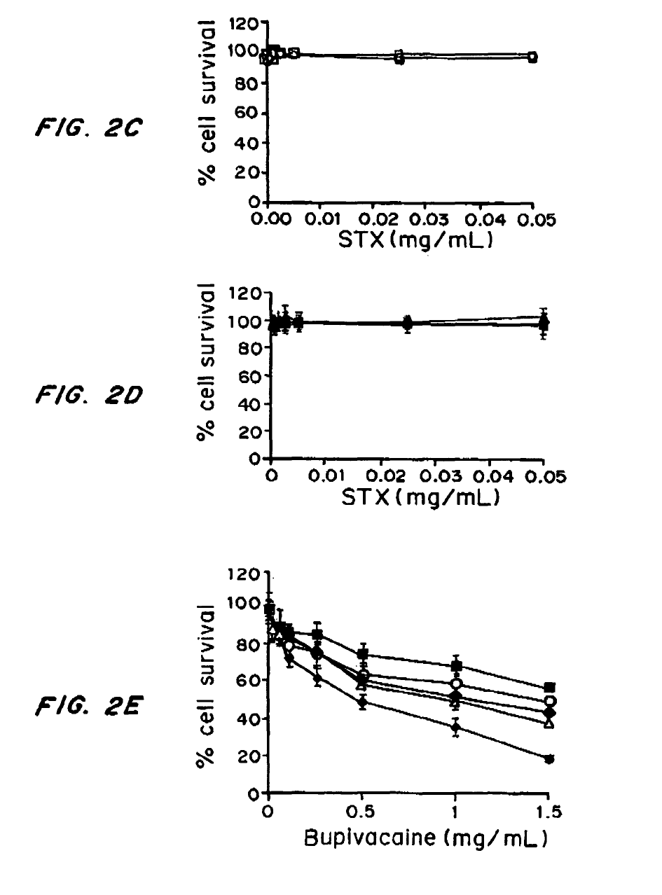

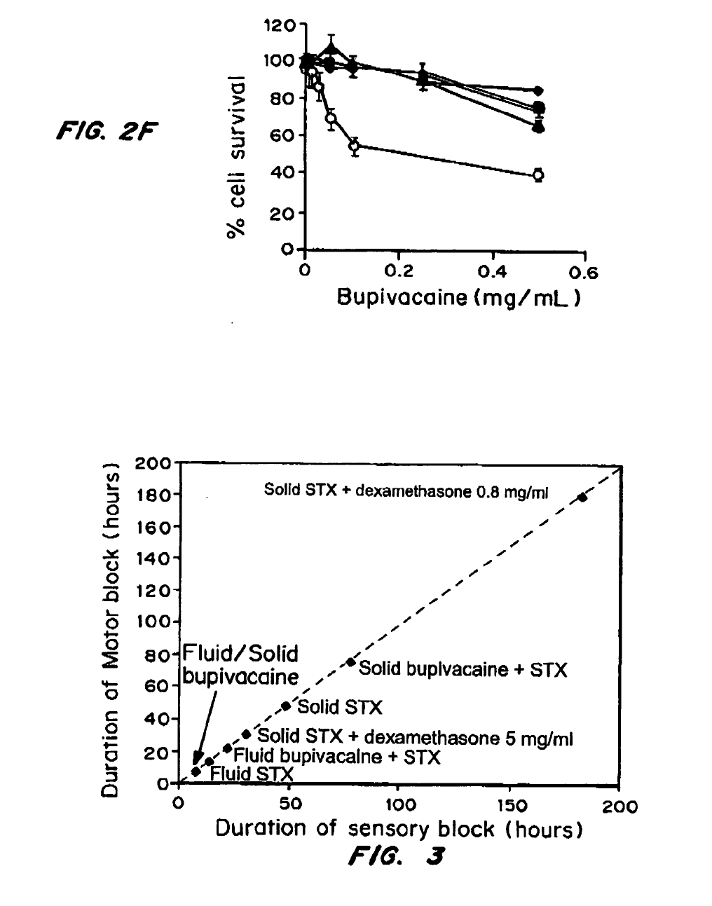

Myotoxicity. Myotoxicity was assessed by exposing C2C12 cells for up to 4 days to a range of concentrations of free compounds (bupivacaine, dexamethasone, or STX), or liposomes with varying drug contents and lipids compositions. Myotoxicity of bupivacaine solution increased with concentration and duration of exposure (FIG. 2A) (Padera, et al. Anesthesiology, 108(5):921-928 (2008)). In contrast, free STX (0.005-0.05 mg/mL) was not myotoxic at any of the concentrations or any durations of exposure tested (e.g., P<0.001, 0.01 versus 0.05 mg/mL at 48 h, and P<0.001 at 0.1 mg/mL at 6 vs. 24 h; FIG. 2C). This concentration range vastly exceeds that required to achieve effective sciatic nerve blockade (Kohane, et al., Reg Anesth Pain Med 25(1):52-59 (2000)). Free dexamethasone (0.005-0.5 mg/mL), singly or in combination with the highest STX concentration (0.05 mg/mL), was not myotoxic at up to 2 days (viability >90%). At day 4, survival was reduced to 80±5% (p<0.05) with 0.5 mg/mL free dexamethasone. This reduction in survival did not occur when the dexamethasone was encapsulated in liposomes.

Blank liposomes were not toxic to cells at concentrations from 0.3 to 6 mg/mL of lipids. Further increasing the lipids reduced cell viability (for example, viability was 50% at 9 mg/mL (p<0.001)). Co-administration of empty liposomes with free drugs did not further reduce cell viability.

Liposomal bupivacaine caused less myotoxicity than the same concentration of free bupivacaine (e.g. p<0.001 at 0.5 mg/mL at 4 days; FIG. 2E). Myotoxicity increased with concentration and duration of exposure (FIG. 2E). Liposomes containing STX, dexamethasone or combinations of both were not myotoxic at any concentration (0-1 mg/mL STX), exposure up to 4 days, or lipid composition. Encapsulation of dexamethasone within bupivacaine liposomes increased toxicity (e.g. p<0.05 at 0.5 mg/mL at 4 days; FIG. 2E), possibly due to the more rapid release of bupivacaine (FIGS. 1A, 1B, 1C). Addition of STX did not increase the myotoxicity of any liposome formulation tested (FIG. 2E).

Neurotoxicity.

Similar studies were performed in PC12 cells (a pheochromocytoma cell line frequently used in neurotoxicity studies (Mearow, et al., Journal of neurochemistry, 83(2):452-462 (2002)). Neurotoxicity of bupivacaine solution increased with concentration and duration of exposure (FIG. 2B). Free or encapsulated STX (0.005-0.05 mg/mL) was not neurotoxic at any concentration or duration of exposure (FIG. 2D), nor was free or encapsulated dexamethasone (0.005-0.05 mg/mL) for up to 5 days. Free dexamethasone (0.05 mg/mL) showed a 20% decrease in survival at day 7. Blank solid and fluid liposomes were not toxic at concentrations from 0.3 to 9 mg/mL. Incorporation of STX into fluid bupivacaine liposomes did not increase their cytotoxicity (FIG. 2F), but incorporation of dexamethasone into bupivacaine liposomes did (e.g. p<0.01 at 0.5 mg/mL FIG. 2F), possibly due to the more rapid release of bupivacaine (FIGS. 1A, 1B, 1C). Similar results were obtained with solid liposomes.

These studies showed that extended exposure to STX-based formulation caused little cytotoxicity, and that therefore the liposomes were good candidates for in vivo use.

Duration of Nerve Blockade

To test the ability of liposomal formulations to produce prolonged nerve blockade and systemic toxicity (increases in latency in the un-injected hindlimb, respiratory distress, death), rats were injected at the sciatic nerve with 0.6 mL of liposome formulations (8 rats per formulation) containing single compounds or combinations. All liposome formulations containing bupivacaine or STX induced motor and sensory nerve block that subsequently reverted to baseline values. Onset of nerve blockade occurred 10-15 min after injection with fluid liposomes, and 0.5-1.5 hr after injection with solid liposomes. The durations of sensory and motor blocks are shown in FIG. 3. These were similar in all cases.

A primary goal of this research was to develop injectable formulations that could achieve reliable and prolonged nerve blockade with STX, or at least without bupivacaine. Polymeric microspheres with TTX alone had been ineffective (Kohane, et al., Pain, 104(1-2):415-421 (2003)). Nerve blockade from fluid liposomes containing STX alone lasted approximately 13.5 hours. Solid liposomes containing STX alone produced even longer blocks, lasting 48 hours, with no signs of systemic toxicity.

Drug interactions that extended the duration of block in polymeric particles containing TTX (Kohane, et al., Pain, 104(1-2):415-421 (2003) also occurred with STX liposomes. The incorporation of some concentrations of dexamethasone into STX liposomes caused marked systemic toxicity and death, presumably because dexamethasone increased liposome permeability to other compounds. For example, STX-containing fluid liposomes with 5 mg/mL of dexamethasone were uniformly fatal, but liposomes containing the same quantities of STX or dexamethasone alone were not toxic. In solid liposomes, co-encapsulation of dexamethasone at 5 mg/mL reduced the duration of block compared to STX (0.031 mg/mL) liposomes (p<0.01), and 2 of 8 animals died. In contrast, dexamethasone at 0.8 mg/mL led to a marked 3.7-fold increase in the duration of nerve blockade (p<0.001), to 180 h or 7.5 days, with no signs of systemic toxicity.

Co-encapsulation of bupivacaine in fluid STX liposomes extended block by 60% to 21.24 h (p<0.001), which was approximately the sum of the block durations of the singly encapsulated compounds. Block from bupivacaine fluid liposomes was 7.3 h. In solid liposomes, co-encapsulation of bupivacaine increased the block duration of STX particles by 56% (p<0.001), which was more than the sum of the durations of block of the individually encapsulated compounds. (Block from solid bupivacaine liposomes was 7.4 h.) There were no signs of systemic toxicity from those formulations.

Empty liposomes and dexamethasone liposomes did not produce nerve blockade during serial testing for 24 hr. No animals developed autotomy (self-injury) of their hindpaws.

Necropsy and Histology

To assess tissue reaction, animals from nerve block experiments were sacrificed 4, 14 and 21 days after injection (n=3 at each time point), if nerve block had resolved. The sciatic nerve and surrounding tissues were harvested, and processed for histology by hematoxylin-eosin staining.

In all eases, liposomes were still seen on gross dissection as a whitish material on the sciatic nerve site at day 4 after injection. Tissues had a benign appearance, with little matting or apparent inflammation. Microscopic examination of these tissues revealed mild to moderate lymphohistiocytic inflammation along the surface of the tissue at 4 days after injection in all samples, which dissipated in all cases by 21 days post-injection. Animals injected with bupivacaine liposomes showed a small number of muscle fibers with nuclear centralization (very mild injury); one had a small area of focal fibrosis. In all other cases, the inflammation did not infiltrate muscle and nerve tissue, and there was no evidence of muscle or nerve damage. Individual particles could not be discerned. As hematoxylin-eosin stained sections are insensitive for identifying nerve injury, Epon-embedded sections in 3 animals in each group were obtained. These did not reveal nerve injury from any formulation at any time point.

Real Time PCR

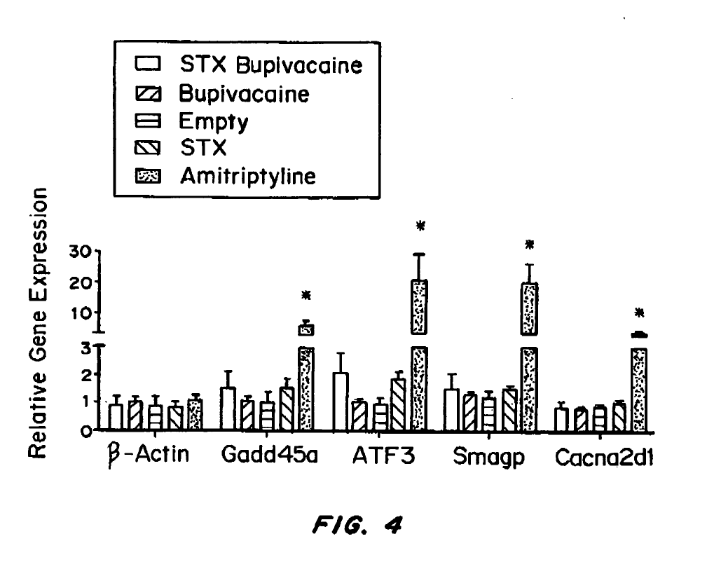

To further assess the presence or absence of nerve injury 4 days after injection, real time PCR was used to study the expression of four genes (Gadd45a (Befort, et al., Eur J Neurosci., 18(4):911-922 (2003)), ATF3 (Nakagomi, et al., J Neurosci., 23(12):5187-5196 (2003); Song, et al., Exp Neural., 209(1):268-278 (2008)), SmagP (Soares, et al., Eur J Neurosci., 21(5):1169-1180 (2005)), Cacna2d1 (Luo, et al., J Pharmacol Exp Ther., 303(3):1199-1205 (2002); Newton, et al., Brain Res Mol Brain Res., 95(1-2):1-8 (2001)) whose expression is altered by nerve injury, using RNA from the dorsal root ganglia of animals that received nerve blocks (n=4 in each group). As a positive control for local anesthetic-associated nerve injury that would be relevant to the formulations used here, 4 animals were given sciatic nerve blocks with 80 mM amitriptyline, a tricyclic antidepressant with local anesthetic properties that resembles bupivacaine in structure and mechanism of action (Gerner, et al., Anesthesiology, 94(4):661-667 (2001)) and that causes severe myotoxicity and neurotoxicity (Lirk, et al., Anesth Analg., 102(6):1728-1733 (2006); Haller, et al., Eur J Anaesthesiol., 24(8):702-708 (2007)). Expression was normalized to GAPDH as an internal control. β-actin was chosen as another gene whose expression should not change with nerve injury. All 4 selected genes were dramatically upregulated in amitriptyline-treated animals 4 days after injection compared to saline-treated controls (FIG. 4, p<0.001). In contrast, these genes were not upregulated by any of the liposomal treatments.

Discussion

A set of drug delivery systems containing the site 1 sodium channel blocker saxitoxin, which can produce prolonged nerve blockade without local toxicity, and in most formulations without systemic toxicity, was tested. They did not require synergistic compounds such as bupivacaine and dexamethasone, although block duration was greatly extended by their use. For example, incorporation of dexamethasone produced blocks lasting more than seven days without any systemic toxicity detectable by neurobehavioral testing methods and clinical exam (Kohane, et al., Pain., 104(1-2):415-421 (2003)). This is in contrast to the experience with polymeric microspheres containing TTX, where particles with the highest non-toxic dose of TTX alone produced a median duration of block of zero minutes, and addition of dexamethasone prolonged their duration of block to only 8 hours. Polymeric microspheres containing TTX also had to be injected with epinephrine to avoid systemic toxicity.

In addition, none of the animals developed autotomy (self-injury) of their hindpaws (Wall, et al., Pain., 7(2):103411 (1979)), a behavior associated with pain which was observed with high prevalence in prolonged blocks from TTX-bupivacaine-dexamethasone polymeric microspheres (Kohane, et al., Pain., 104(1-2):415-421 (2003)). Importantly for the potential clinical applicability of prolonged duration local anesthetics, this implies that autotomy is not an unavoidable concomitant of prolonged nerve blockade. It is not clear why autotomy was not seen here. Differences between the liposomes and PLGA particles per se would be expected to have effects at the site of injection at the hip, whereas autotomy occurs at the paw (Wall, et al., Pain., 7(2):103-111 (1979)).

Block from the bupivacaine liposomes used here lasted 7 hours. Co-injection of solutions containing site 1 sodium channel blockers and “conventional” local anesthetics prolongs block showed a 6 fold increase compared to the compounds injected separately (Barnet, et al., Pain., 110(1-2):432-438 (2004)); this also occurs when they are co-encapsulated in polymeric microspheres (Kohane, et al., Pain., 104(1-2):415-421 (2003)). Prolongation was also seen, but of lesser magnitude. This difference may be because co-encapsulation of bupivacaine in saxitoxin-containing liposomes reduced the loading of the latter compound (TABLE 1).

There was mild to moderate focal inflammation 4 days after local injection of liposomes that was completely resolved by 21 days. No myotoxicity or neurotoxicity was seen in any formulations that did not contain bupivacaine. The finding that there was no nerve injury was supported by the PCR analysis of genes that are known to be related to nerve injury (FIG. 4). These findings are a great improvement over those from polymeric and/or bupivacaine-containing particles in terms of myotoxicity and inflammation.

This improvement in tissue reaction was surprising given previous experience that injury results from controlled release of local anesthetics using a wide range of delivery vehicles. Although the reason for this improvement is not clear, it confirms the observation that the presence of particles per se—and by inference their nature—has a large impact on local injury (Padera, et al., Anesthesiology., 108(5):921-928 (2008)).

Aside from the formulations where high dexamethasone contents increased efflux of STX, there was no systemic toxicity from STX-containing liposomes, even with prolonged durations of block, as assessed by the absence of changes in latency in the contralateral extremity. The latter is a validated measure of systemic distribution and toxicity of local anesthetics (Kohane, et al., Anesthesiology., 89(1):119-131 (1998); Kohane, et al., Reg Anesth Pain Med., 26(3):239-245 (2001)). This is important since with polymeric particles, useful blockade could not be achieved without initial signs of systemic toxicity, due to burst release of TTX. This toxicity occurred even in the presence of epinephrine in the injectate, while here there was no systemic toxicity even without epinephrine.

Another significant advantage of this formulation over the polymeric systems (Kohane, et al., Pain, 104(1-2):415-421 (2003)) is the very small coefficient of variation in block duration. For example, STX+dexamethasone liposomes gave a block duration of 180±4 hours (coefficient of variation=2.2%). In contrast, 60 μm polymeric particles gave a median block duration of 9.25 days, with an interquartile range of 8.3-14.8 days (Kohane, et al., Pain, 104(1-2):415-421 (2003)). This lower variability may be because the liposomes were a better suspension, without needle-clogging. Reproducibility of block duration is an important clinical performance criterion.

In conclusion: ultra long-acting toxin-based liposomal local anesthetics were developed that were biocompatible in terms of myotoxicity, neurotoxicity, inflammation, and systemic toxicity, unlike the toxins encapsulated in polymeric microparticles and did not cause autotomy in an animal model system.

We claim:

1. A composition for rapid onset nerve blockade, consisting of a site I sodium channel blocker, optionally in combination with a glucocorticoid, in a liposome, wherein the composition is effective to provide reliable prolonged nerve blockade in the absence of local toxicity relative to the site I sodium channel blocker encapsulated in a polymeric microparticle.

2. The composition of claim 1 wherein the site 1 sodium channel blocker is selected from the group consisting of tetrodotoxin (TTX), saxitoxin (STX), decarbamoyl saxitoxin, neosaxitoxin, and the gonyautoxins.

3. The composition of claim 2 comprising an effective amount of a glucocorticoid selected from the group consisting of dexamethasone, cortisone, hydrocortisone, prednisone, beclomethasone, betamethasone, flunisolide, methyl prednisone, para methasone, prednisolone, triamcinolome, alclometasone, amcinonide, clobetasol, fludrocortisone, difluorosone diacetate, fluocinolone acetonide, fluoromethalone, flurandrenolide, halcinonide, medrysone, and mometasone, and pharmaceutically acceptable salts and mixtures thereof, wherein the glucocorticoid enhances nerve block in the absence of local toxicity relative to the site 1 sodium channel blocker alone.

4. The composition of claim 3 wherein the site 1 sodium channel blocker is STX and the glucocorticoid is dexamethasone.

5. The composition of claim 1 wherein the onset of nerve block is between 10 and 120 min.

6. The composition of claim 1 where in the onset of nerve block is between 10 and 15 min.

7. The composition of claim 1 wherein the liposome is a solid liposome.

8. The composition of claim 1 wherein the liposome is a fluid liposome.

9. A method for rapid onset nerve blockade, comprising administering an effective amount of a composition consisting of a site I sodium channel blocker, optionally in combination with a glucocorticoid, in a liposome, to provide reliable prolonged nerve blockade in the absence of local toxicity.

10. The method of claim 9 wherein the site 1 sodium channel blacker is selected from the group consisting of tetrodotoxin (TTX), saxitoxin (STX), decarbamoyl saxitoxin, neosaxitoxin, and the gonyautoxins.

11. The method of claim 9 wherein the composition comprises an effective amount of a glucocorticoid selected from the group consisting of dexamethasone, cortisone, hydrocortisone, prednisone, beclomethasone, betamethasone, flunisolide, methyl prednisone, para methasone, prednisolone, triamcinolone, alclometasone, amcinonide, clobetasol, fludrocortisone, difluorosone diacetate, fluocinolone acetonide, fluoromethalone, flurandrenolide, halcinonide, medrysone, and mometasone, and pharmaceutically acceptable salts and mixtures thereof, to enhance nerve block in the absence of local toxicity as compared to the sodium channel blocker alone.

12. The method of claim 11 wherein the site 1 sodium channel blocker is STX and the glucocorticoid is dexamethasone.

13. The method of claim 9 wherein the onset of nerve block is between 10 and 120 min.

14. The method of claim 9 where in the onset of nerve block is between 10 and 15 min.

15. The method of claim 9 wherein the liposome is a solid liposome.

16. The method of claim 9 wherein the liposome is a fluid liposome.
